(19) United States
(12) Patent Application Publication
(10) Pub. No.: US 2025/0152080 A1
(43) Pub. Date: May 15, 2025

(54) DISTRIBUTED WIRELESS HIGH-DENSITY SURFACE ELECTROMYOGRAPHY SYNCHRONOUS ACQUISITION SYSTEM

(71) Applicant: DALIAN UNIVERSITY OF TECHNOLOGY, Dalian (CN)

(72) Inventors: Liming SHU, Dalian (CN); Hong TANG, Dalian (CN); Taiyang ZHAO, Dalian (CN)

(21) Appl. No.: 19/028,059

(22) Filed: Jan. 17, 2025

Related U.S. Application Data
(63) Continuation of application No. PCT/CN2024/084721, filed on Mar. 29, 2024.

(51) Int. Cl. A61B 5/397; A61B 5/00; A61B 5/11; A61B 5/257; A61B 5/263; A61B 5/296; A61B 5/313; G16H 40/67

(52) U.S. Cl. CPC A61B 5/397; A61B 5/1126; A61B 5/257; A61B 5/263; A61B 5/296; A61B 5/313; A61B 5/681; A61B 5/7207; A61B 5/7225; A61B 5/742; G16H 40/67; A61B 2560/0214; A61B 2560/045; A61B 2562/0209; A61B 2562/0219; A61B 2562/046; A61B 2562/222; A61B 2562/227

(57) ABSTRACT

The present invention belongs to the field of human-computer interaction, and discloses a distributed wireless high-density surface electromyography synchronous acquisition system, comprising high-density electromyography front-end acquisition control units, a base station unit and a host computer, wherein data is transmitted between the high-density electromyography front-end acquisition control units and the base station unit through a wireless system; each high-density electromyography front-end acquisition control unit comprises a high-density electromyography acquisition array, a reference electrode and a signal transmission module; the base station unit comprises a synchronization control module, a wireless routing module and a charging and discharging module; the host computer is a client computer program, which is loaded with a signal preprocessing and display module and a high-density electromyography decoding algorithm module; and information received from the base station unit is acquired and analyzed and results are displayed by a client.

### TECHNICAL FIELD

The present invention belongs to the field of human-computer interaction, and particularly relates to a human body surface physiological electromyogram acquisition, transmission and analysis system, in particular to a distributed high-density surface electromyography data wireless synchronous acquisition and analysis system.

### BACKGROUND

A Surface Electromyogram (sEMG) is a bioelectrical signal generated by human neuromuscular activity, which can reflect an important physiological state and health information of the human body, and has the advantages such as being non-invasive, non-traumatic and easy to operate. Surface electromyograms are usually extracted by single or double electrodes placed at the muscle bellies. However, the internal activation state of a skeletal muscle is uneven during exercise, and when signal extraction is conducted with relatively few electrode channels, differences will be caused in signals due to different positions of the electrode(s). Therefore, it is difficult to perform the tasks such as accurate muscle activation analysis and muscle force estimation. Two-dimensional matrix surface electrodes are used by High-Density Surface Electromyography (HD-sEMG) to cover an entire muscle skin area, so that time and frequency domain as well as spatial domain information of a target muscle can be obtained by high-density surface electromyography, thus to obtain a visual map of the skeletal muscle relative to an activated area, which overcomes the disadvantages of low spatial resolution of ordinary electrodes. As a means of objectively quantifying a human body physiological electrical signal, a high-density surface electromyogram has a wide application prospect in human factors engineering, virtual reality, rehabilitation medicine and sports training, in addition to a wide range of engineering applications in the field of human-computer interaction intention recognition such as prosthetic control.

With respect to the needs of real-time synchronous acquisition of multiple motion scenarios and multiple parts of the human body, the shortcomings of the existing surface electromyogram acquisition technology are mainly reflected in the following two aspects:
(1) With respect to the wireless requirements of high-density surface electromyography data acquisition, it is difficult to take account of the number of channels, sampling frequency and synchronizability simultaneously in a traditional wireless acquisition device.

Li Sujiao et al. have designed an array-type high-density surface electromyography acquisition device, wherein original electromyograms are acquired by a self-made 16-channel array-type high-density surface electromyography electrode slice and converted into digital signals by a multi-channel signal processing module, and a wireless communication module based on Bluetooth is used for multi-channel digital signal transmission, which can realize the acquisition of 16-channel electromyograms. Sheng Xinjun et al. have designed a surface electromyogram acquisition system for decoding motor units, which can complete the acquisition of 4-channel electromyograms by using a self-made electrode. The above methods have taken into account the convenience of wearing the device, but the number of channels of the device for acquiring electromyograms is small, and signals of multiple muscle groups cannot be acquired jointly, which are limited in practical application.
(2) In an existing high-density surface electromyography acquisition scheme, a hardware circuit is complicated, numerous components are introduced, and a high-density electromyography circuit is bulky after integration, which is difficult to meet the requirement of being wearable and cannot meet the requirement of real-time synchronous measurement at multiple measurement points during a motion of the human body, and the problem of poor human body adaptability in a complex motion environment is found.

Zhang Hengyi et al. have designed a high-density active flexible electrode array and a signal conditioning circuit thereof, which uses a two-stage amplifier circuit to amplify and filter electromyograms, and can accurately measure the electromyograms of a single channel when cooperated with an NI acquisition card. Feng Wanyu et al. have designed an electromyography acquisition device based on flexible active electrodes, which uses an active circuit to conduct impedance conversion and channel filtering of an acquisition channel, and can accurately extract electromyograms of a single channel from four electrodes. Both of the above schemes are focused on acquisition of accurate single-channel electromyograms and accurate measurement of voltage amplitude of the electromyograms by setting resistive capacitance and impedance values, while the existing acquisition scheme is difficult to accurately measure multi-channel electromyograms.

In view of the above problems, the present invention aims to develop a novel wireless high-density surface electromyography data acquisition system, which has a distributed modular design, and acquisition front ends are separated from a signal storage module to ensure the ease of use and low coupling of the device; completes a comprehensive optimization design of an internal signal acquisition circuit to achieve high-quality electromyography data acquisition; has an optimized real-time transmission and system synchronization function, and has the characteristics such as large number of channels, high sampling frequency and strong synchronization; has a lightweight structural design and good wearability to ensure stable serviceability of the acquisition device in multiple scenarios; and has a synchronous trigger function design, and can realize synchronous acquisition with other acquisition devices such as a motion capture device and an electroencephalograph system.

### SUMMARY

The present invention aims to solve the problem of poor signal acquisition and use experiences in the electromyography acquisition technology, such as poor quality of signals acquired, limited number of channels and insufficient synchronizability; provide a new scheme for high-density surface electromyography distributed wireless measurement; improve the accuracy of high-density surface electromyograms acquired; and broaden the application scenarios of high-density surface electromyography in brain-computer interface, human factors engineering, virtual reality, rehabilitation medicine and sports training.

The technical solution of the present invention is as follows:

A distributed wireless high-density surface electromyography synchronous acquisition system comprises high-density electromyography front-end acquisition control units, a base station unit and a host computer, wherein data is transmitted between the high-density electromyography front-end acquisition control units and the base station unit through a wireless system; each high-density electromyography front-end acquisition control unit comprises a high-density electromyography acquisition array, a reference electrode and a signal transmission module; the base station unit comprises a synchronization control module, a wireless routing module and a charging and discharging module; the host computer is a client computer program, which is loaded with a signal preprocessing and display module and a high-density electromyography decoding algorithm module; and information received from the base station unit is acquired and analyzed and results are displayed by a client;

When used, the reference electrode is moistened and fixed to a skeletal muscle tendon of a human body, the high-density electromyography acquisition array is attached to a skeletal muscle belly of the human body, and the reference electrode and the high-density electromyography acquisition array are connected with the signal transmission module, respectively; after an acquisition instruction is issued by the host computer, the instruction is analyzed by the base station unit to control the high-density electromyography front-end acquisition control units to start working; skin surface electromyograms are transmitted to the signal transmission module by the high-density electromyography acquisition array, signals in the same time period are packaged by the signal transmission module and sent to the base station unit, signals transmitted by multiple high-density electromyography front-end acquisition control units are integrated by the base station unit and transmitted to the host computer through a data line in real time; and files are displayed and stored by the host computer.

Further, the high-density electromyography acquisition array is composed of immersion gold contacts, a copper flat cable and a polyimide substrate, the immersion gold contacts are located on the polyimide substrate and led out by the copper flat cable; the high-density electromyography acquisition array is distributed and fixed to each skeletal muscle belly of the human body by foam double-sided adhesive tape, wherein a hole position is preserved for medical foam double-sided adhesive tape and filled with conductive paste, the contacts are in contact with human skin through the conductive paste, and the skin surface electromyograms are transmitted to the signal transmission module through the conductive paste, the contacts and the copper flat cable; the size and spacing of the contacts of the high-density electromyography acquisition array can be freely customized according to a muscle shape, and the maximum number of contacts in the same high-density electromyography acquisition array is 64; and the immersion gold contacts on the high-density electromyography acquisition array are arranged according to the muscle shape on the basis of ensuring a consistent spacing. The contacts adopt an immersion gold process to improve corrosion resistance of the contacts against sweat and ensure stability of the electromyograms acquired; The polyimide substrate has the characteristics such good commonness with skin and long-term contact with the skin, which can integrate the contacts and the flat cable together well to form the high-density electromyography acquisition array.

Further, the reference electrode is fixed to the muscle tendon of the human body and is designed into a wristband type; and one reference electrode is shared by the multiple high-density electromyography front-end acquisition control units.

Further, the signal transmission module comprises a high-density electromyography acquisition array connector, an electromyogram processing chip, a microprocessor, a six-axis acceleration sensor, a power supply control module and a battery module; the high-density electromyography acquisition array is connected by the high-density electromyography acquisition array connector to transmit original electromyograms to the signal transmission module; the electromyogram processing chip is used for amplifying and sampling, bandpass filtering and analog-to-digital conversion of the electromyograms, the resolution is 16 bits and can be extended to 24 bits, and data sampling of 64-channel signals up to 30 KSps can be completed; the microprocessor adopts a CC3235S chip integrated with a dual-band Wi-Fi low-power 5G communication chip to conduct multi-channel data integration of received high-density electromyograms and acceleration signals and real-time control of synchronization signal transmission, and finally realize lossless data processing and low-power data communication; a three-axis acceleration sensor and a three-axis gyroscope are integrated in the six-axis acceleration sensor to obtain motion data during human body electromyography data acquisition; and the power supply control module and the battery module are used for supplying power to the high-density electromyography front-end acquisition control units and protect the high-density electromyography front-end acquisition control units from electrical underload or overload, and a low dropout regulator is designed to reduce background noise in the electromyograms;

The skin surface electromyograms are transmitted to the signal transmission module by the high-density electromyography acquisition array and converted into digital signals with a resolution of 24 bits by the electromyogram processing chip; and the acceleration signals are transmitted to the microprocessor by the six-axis acceleration sensor, the high-density electromyograms and the acceleration signals are simultaneously received by the microprocessor, and the signals in the same time period are packaged and sent to the base station unit.

Further, a timing pulse is sent out by the synchronization control module in the form of broadcast to inform all high-density electromyography front-end acquisition control units to conduct synchronous calibration; the pulse interval is controlled at about 1 second, which can ensure that the time difference among the front-end acquisition control units at a sampling moment is less than 1 millisecond in a whole sampling process; the synchronization control module is provided with a synchronous input port and a synchronous output port, wherein the synchronous input port is based on an signal input of an external device to start the wireless high-density surface electromyography synchronous acquisition system and complete synchronous acquisition with the external device; and when an acquisition start command is issued by the host computer, a pulse will be synchronously output by the synchronization control module to start the external device through the synchronous output port.

Further, the wireless routing module is used for receiving wireless signals transmitted by the high-density electromyography front-end acquisition control units and sending wireless control signals to the high-density electromyography front-end acquisition control units; and the electromyograms are transmitted to the host computer after being obtained by the wireless routing module through a wired network.

Further, the charging and discharging module comprises a power supply control module and a lithium-ion battery, and is mainly used for charging the high-density electromyography front-end acquisition control units; and the power supply control module mainly plays the roles of reducing voltage and stabilizing current, and the lithium-ion battery is used for storing electric energy to provide energy guarantee for the system when an external power supply is not available.

Further, the host computer is a computer program which integrates the functions of storage, calculation and display, and comprises the signal preprocessing and display module and the high-density electromyography decoding algorithm module; preprocessing of spectrum analysis and display, high and low pass filtering processing, motion artifact elimination, bad track detection and damaged data elimination of high-density data are realized by the signal preprocessing and display module, and the data is transmitted to the high-density electromyography decoding algorithm module; and preprocessed high-density electromyograms are decomposed by the high-density electromyography decoding algorithm module based on a Blind Source Separation (BSS) algorithm, and activation and discharge time series innervating motor neurons in corresponding motions are extracted.

The distributed wireless high-density surface electromyography synchronous acquisition system established can simultaneously acquire electromyography data of up to 640 channels in real time.

A distributed wireless high-density surface electromyography synchronous acquisition device comprises:

A high-density electromyography front-end acquisition control unit which comprises a high-density electromyography acquisition array 0101, a signal transmission module 0102 and a reference electrode 0103; and the high-density electromyography acquisition array 0101 is connected with the signal transmission module 0102 through a high-density electromyography acquisition array connector 0102-1, and medical foam double-sided adhesive tape 0101-1 matched with the high-density electromyography acquisition array is arranged at the bottom of the high-density electromyography acquisition array 0101;

The signal transmission module 0102 comprises the high-density electromyography acquisition array connector 0102-1, an acquisition control unit integrated circuit board 0102-2, a charging contact 0102-3, a lithium ion rechargeable battery 0102-4, a reference electrode interface 0102-5 and a signal indicator light 0102-6; and a circuit part of the acquisition control unit integrated circuit board 0102-2 comprises an electromyogram processing chip, a six-axis acceleration sensor, a microprocessor and a power supply management module, which are designed by copper paving of a four-layer board;

A base station unit 0200 which comprises a storage box 0201, a display screen 0202, a charging case signal light 0203, a charging case 0204, a synchronous input interface 0205, a synchronous output interface 0206, a host computer communication network interface 0207, a base station unit charging interface 0208, a base station unit switch 0209, a display screen switch 0210 and base station unit power indicator lights 0211; the storage box 0201 is used for accommodating a data line and the high-density electromyography acquisition array; the display screen 0202 is used for displaying operating status information, power consumption and signal connection strength of each signal transmission module, as well as remaining power of the base station unit; the charging case signal light 0203 is used for displaying the power condition of the signal transmission module; the charging case 0204 is used for charging and accommodating the signal transmission module, and the signal transmission module can be fixed by a magnetic design in the case to ensure smooth charging of the module; the synchronous input interface 0205 is connected with an external device through a BNC q9 connector to cooperate with a peripheral device to conduct synchronous acquisition; the synchronous output interface 0206 is connected with the peripheral device to control the peripheral device to conduct synchronous acquisition; the host computer communication network interface 0207 is used for transmitting data acquired to the host computer and receiving control instructions from the host computer, which adopts a Modbus communication protocol in RS485 form and works in a full-duplex mode; the base station unit charging interface 0208 can be connected to an AC power supply to charge the base station unit; the base station unit switch 0209 is used for controlling the start and stop of the base station unit; the display screen switch 0210 is used for independently controlling the on-off of the display screen; and the base station unit power indicator lights 0211 are used for displaying the remaining power of the base station unit in real time, when four LED lights are on, it indicates that the remaining power of the base station unit is 100%, and when one LED light is off, it means that the power is reduced by 25%;

The host computer is loaded with a data preprocessing and decoding algorithm.

The present invention has the following beneficial effects:
(1) Synchronous acquisition of high-quality signals in multiple scenarios: the distributed high-density surface electromyography synchronous acquisition system established adopts a wireless distributed modular design and a low noise circuit layout to realize combined acquisition of electromyograms of multiple different muscle groups with a high signal-to-noise ratio, a multi-channel and a high sampling rate.
(2) Synchronous acquisition of multi-modal data: the acceleration signals and high-density electromyography acquisition array are integrated into one module by a front-end acquisition system to complete synchronous acquisition of motion data and high-density electromyography data, and the system also supports synchronous input and output trigger acquisition to realize synchronous acquisition with an external device.
(3) Realization of data visualization: the present invention adopts a composite base station design to synchronously integrate multi-channel electromyograms and stream the multi-channel electromyograms to a host computer by Wi-Fi. A low-latency signal display function can be provided in combination with interactive UI data analysis software.
(4) Development of a signal analysis tool: the present invention designs a signal analysis and processing module to decode the electromyograms through the steps such as signal preprocessing, channel screening, blind source separation and clustering analysis, and provide an interpretable tool for neuromuscular analysis.

### DESCRIPTION OF DRAWINGS

FIG. 1 is a frame schematic diagram of a distributed wireless high-density surface electromyography synchronous acquisition system;

FIG. 2 is a schematic diagram of a high-density electromyography front-end acquisition control unit;

FIG. 3 is a frame diagram of a hardware circuit in a signal transmission module of a high-density electromyography front-end acquisition control unit;

FIG. 4 is a composition schematic diagram of a base station unit;

FIG. 5 is an outside view of a high-density electromyography front-end acquisition control unit, wherein (a) is an overall configuration diagram of a high-density electromyography front-end acquisition control unit, (b) is a front view of a signal transmission module, and (c) a rear view of a signal transmission module;

FIG. 6 is an outside view of a base station unit, wherein (a) is an overall outside view of a base station unit, and (b) is structural diagram of a function panel of a base station unit; and

FIG. 7 is a schematic diagram of a data preprocessing and decoding algorithm of a distributed wireless high-density surface electromyography synchronous acquisition system, wherein (a) is a principle block diagram of a data preprocessing and display module, and (b) is a principle block diagram of a high-density electromyography decoding algorithm module.

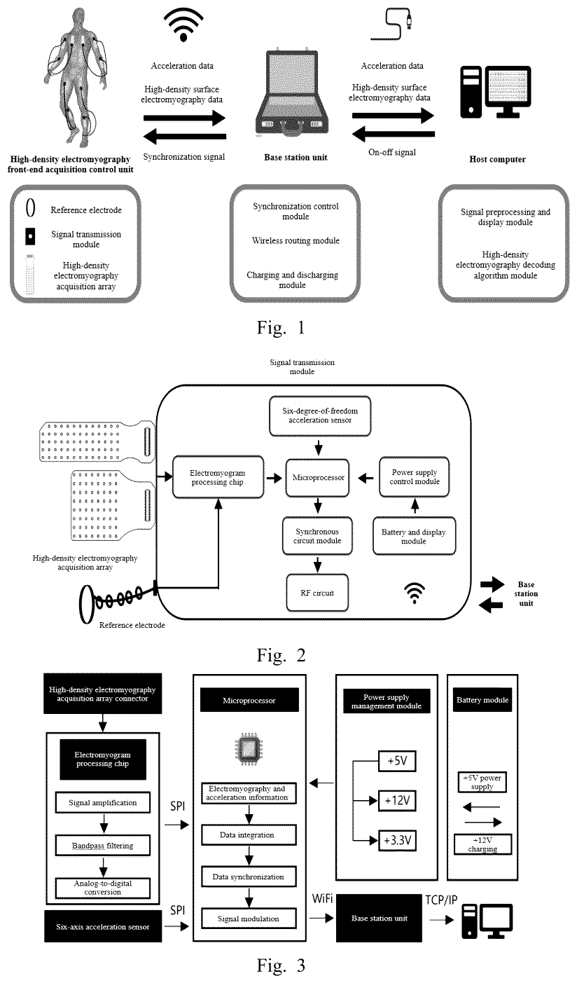

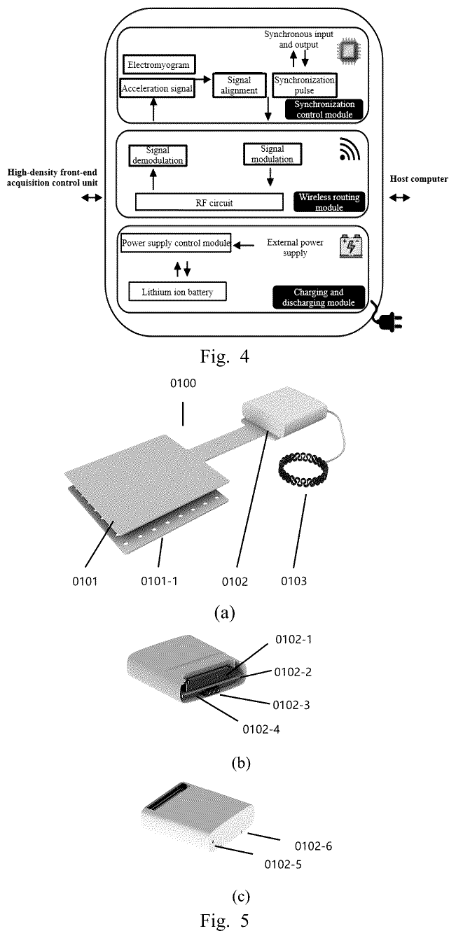

In the drawings: 0101 high-density electromyography acquisition array, comprising immersion gold contacts, a copper flat cable and a polyimide substrate, 0101-1 medical foam double-sided adhesive tape matched with the high-density electromyography acquisition array, 0102 signal transmission module, 0103 reference electrode, 0102-1 high-density electromyography acquisition array connector, 0102-2 acquisition control unit integrated circuit board, 0102-3 charging contact, 0102-4 lithium ion rechargeable battery, 0102-5 reference electrode interface, and 0102-6 signal indicator light; 0200 base station unit, 0201 storage box, 0202 display screen, 0203 charging case signal light, 0204 charging case, 0205 synchronous input interface, 0206 synchronous output interface, 0207 host computer communication network interface, 0208 base station unit charging interface, 0209 base station unit switch, 0210 display screen switch, and 0211 base station unit power indicator light.

### DETAILED DESCRIPTION

Specific embodiments of the present invention are further described below in combination with the drawings and the technical solution.

A distributed wireless high-density surface electromyography synchronous acquisition system realizes a miniaturized structure design by signal transmission modules in high-density electromyography front-end acquisition control units, and ensures the comfort of wear on a human body; A base station unit is designed into a box structure, which integrates a synchronization control module, a wireless routing module and a charging and discharging module, and when the system is not in use, the high-density electromyography front-end acquisition control units can be accommodated in the base station unit to form an integrated structure; and the host computer is a client computer program, which is loaded with a signal preprocessing and display module and a high-density electromyography decoding algorithm module, and an overall frame schematic diagram is shown in FIG. 1.

When used, a reference electrode is moistened and fixed to a skeletal muscle tendon of the human body, the high-density electromyography acquisition array is attached to a skeletal muscle belly on the surface of the human body, and the reference electrode and the high-density electromyography acquisition array are connected with the signal transmission modules to complete the installation of the high-density electromyography front-end acquisition control units.

After an acquisition instruction is issued by the host computer, the instruction is analyzed by the base station unit to control the high-density electromyography front-end acquisition control units to start working. Skin surface electromyograms are transmitted to the signal transmission modules by the high-density electromyography acquisition array and converted into digital signals with a resolution of 24 bits by the electromyogram processing chip; and acceleration signals are transmitted to the microprocessor by a six-degree-of-freedom acceleration sensor. High-density electromyograms and six-axis acceleration signals are simultaneously received by the microprocessor, and the signals in the same time period are packaged and sent to the base station unit. Signals transmitted by multiple front-end acquisition control units are integrated by the base station unit and transmitted to the host computer through a data line in real time. A data waveform is displayed by the host computer in real time. After a data storage function is enabled, data is periodically transmitted to a file in a specified path.

According to the characteristics of high-fidelity signal acquisition of the system, a scheme of preprocessing analog signals input by high-density electromyography acquisition front ends is proposed to ensure the quality of high-density electromyogram and acceleration signal acquisition. The purity of the analog signals is controlled by the high-density electromyography acquisition front ends through constructing an acquisition method suitable for long-term stable wear, integrating a power management module and designing a circuit architecture for analog-digital separation.

### (1) Design of High-Density Electromyography Front-End Acquisition Control Unit

In the present embodiment, an outside view of a high-density electromyography front-end acquisition control unit is shown in FIG. 5.

A high-density electromyography acquisition array 0101 is manufactured on the basis of a flexible material, can be tightly fitted to skin surface, and can complete the conduction of 64-channel electromyography voltage signals.

After a preserved hole position of medical foam double-sided adhesive tape 0101-1 is filled with conductive paste, the tape is closely fitted to the high-density electromyography acquisition array 0101 and the skin surface, and long-term wear of the high-density electromyography front-end acquisition control units can be realized.

Electromyograms in the form of voltage are converted into digital electromyograms in a computer by a signal transmission module 0102, and the digital electromyograms are uploaded to the base station unit.

The high-density electromyography acquisition array 0101 is connected with the signal transmission module 0102 through a high-density electromyography acquisition array connector 0102-1, which is convenient for the wear of the high-density electromyography front-end acquisition control units.

A circuit part of an acquisition control unit integrated circuit board 0102-2 comprises an electromyogram processing chip, a six-degree-of-freedom acceleration sensor, a microprocessor and a power supply management module, which are designed by copper paving of a four-layer board to reduce signal interference between channels. The electromyogram processing chip is used for amplifying and filtering 64-channel original electromyograms, and conducting analog-to-digital conversion with a resolution of 24 bits; the six-degree-of-freedom acceleration sensor is used for acquiring acceleration information of human body torso, which provides accurate and comprehensive data for real-time monitoring and recording of postures and motion tracks of an individual; the microprocessor is used for communicating with the base station unit in real time through on-board Wi-Fi, packaging and uploading the electromyograms, and receiving and executing control instructions from the base station unit at the same time; and the power supply management module is integrated with a low voltage difference linear voltage regulator circuit with a rectification function to ensure the voltage stability of a power supply by adjusting the linear output a P-MOS, and minimize the power frequency interference from the power supply.

A lithium-ion rechargeable battery 0102-4 is used for supplying power to the high-density electromyography front-end acquisition control units to ensure that an acquisition device can work continuously for more than four hours or stand by for more than ten hours.

A reference electrode interface 0102-5 adopts a hot-swappable wiring terminal to fix a leading wire of the reference electrode, the other end of the leading wire is connected with an electromyography wristband, the electromyography wristband is moistened by water containing impurities to conduct electricity, a soft, breathable and absorbent fabric belt is selected to ensure wearing comfort and electrode conductivity, and a multi-button design of the wristband ensures that multiple reference electrode leading wires can be connected with the same wristband.

A signal indicator light 0102-6 can be used for displaying the operating status of the signal transmission module, a red light flashes when the device is in a charging state, a green light is steady on when the device is in a standby state, and the green light flashes when the device is in a working state.

### (2) Design of Base Station Unit

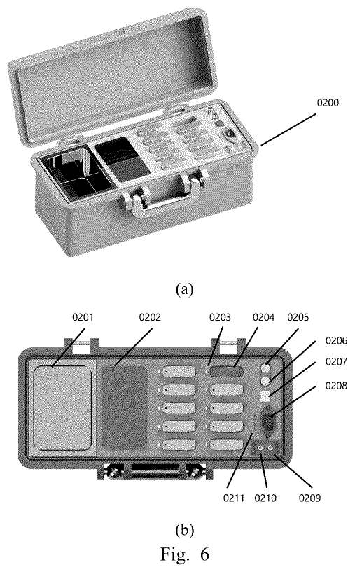

In the present embodiment, an outside view of a base station unit 0200 is shown in FIG. 6.

A storage box 0201 can be used for accommodating a data line and the high-density electromyography acquisition array. A display screen 0202 can be used for displaying device status information, such as operating status information, power consumption and signal connection strength of each signal transmission module, as well as remaining power of the base station unit. A charging case signal light 0203 can be used for displaying the power condition of the signal transmission module.

A charging case 0204 is used for charging and accommodating the signal transmission module, and the signal transmission module can be fixed by a magnetic design in the case to ensure smooth charging of the module.

A synchronous input interface 0205 is connected with an external device through a BNC q9 connector to cooperate with a peripheral device to conduct synchronous acquisition.

A synchronous output interface 0206 is connected with the peripheral device to control the peripheral device to conduct synchronous acquisition.

A host computer communication network interface 0207 is used for transmitting data acquired to the host computer and receiving control instructions from the host computer, which adopts a Modbus communication protocol in RS485 form and works in a full-duplex mode to ensure flexibility in data exchange.

A base station unit charging interface 0208 can be connected to an AC power supply to charge the base station unit; a base station unit switch 0209 is used for controlling the start and stop of the base station unit; a display screen switch 0210 is used for independently controlling the on-off of the display screen; and base station unit power indicator lights 0211 are used for displaying the remaining power of the base station unit in real time, when four LED lights are on, it indicates that the remaining power of the base station unit is 100%, and when one LED light is off, it means that the power is reduced by 25%.

The base station unit supports simultaneous data transmission with 10 high-density front-end acquisition control units, and each high-density front-end acquisition control unit is capable of acquiring electromyograms of up to 64 channels and acceleration signals of 6 channels, i.e., the base station unit is capable of simultaneously transmitting electromyograms of 640 channels and acceleration signals of 60 channels; and the base station unit also supports the device control communication between the host computer and a slave computer, which can meet the peak transmission bandwidth requirement of 300 KSps.

### (3) Functional Design of System Software of Host Computer

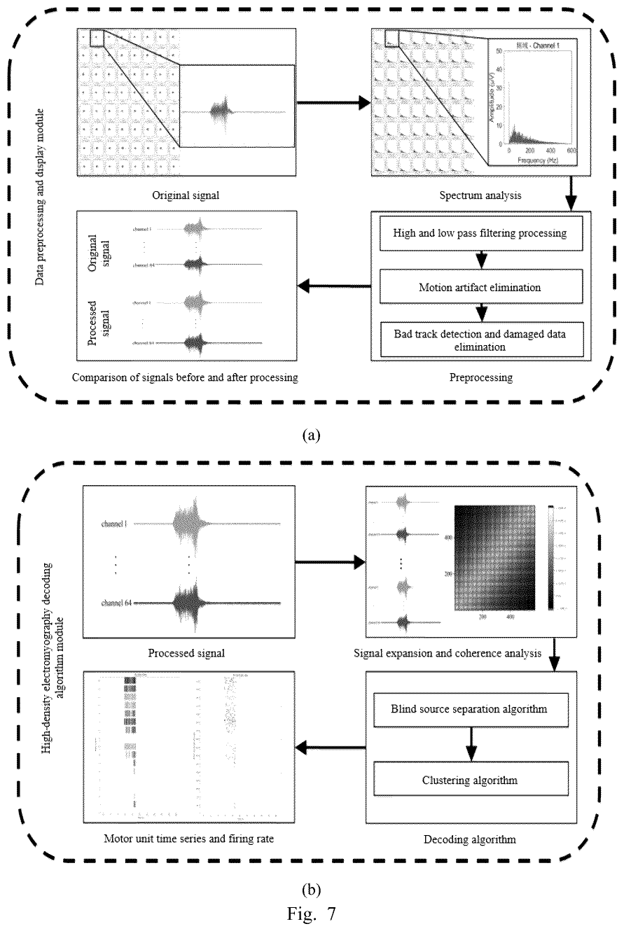

In the present embodiment, a data preprocessing and decoding algorithm loaded in the host computer is shown in FIG. 7.

Data processed by the signal transmission module is transmitted to the host computer in real time through a network line, data obtained is displayed by system software in real time, and data acquired is displayed and stored in a specified file folder. Subsequent processing of the signals can be conducted by the algorithm module loaded in the system software, and analysis results can be presented in a user graphical interface.

In the present invention, a system work flow is as follows:

After the system software is started, ports of the base station unit are found according to prompts of the software, and the signal transmission module is taken out from the base station unit and connected to the high-density electromyography acquisition array and the reference electrode, and electromyography data acquisition can be conducted after solid connection is ensured. Before data is formally stored, a waveform window is opened first, a matching degree between a waveform of electromyograms acquired and an action is observed to adjust the wearing condition of the device, and data recording can be started when the waveform shows a fluctuation following the action and a low amplitude in a resting state.

A user can manage and play back a file saved through the system software of the host computer.

### (1) Data Preprocessing and Display Module

The original electromyograms acquired by the high-density electromyography front-end acquisition control units contains many noise components, such as motion artifacts, system background noise, human electrocardiogram, etc. The noise of this part is mainly concentrated in a low frequency band, and most noise interference can be eliminated by a filtering module. Anti-aliasing filtering is conducted by the filter module at a sampling frequency of 2000 Hz, and then 20 Hz high-pass second-order Butterworth filtering and 500 Hz low-pass second-order Butterworth filtering are conducted to retain the electromyograms at a target frequency band of 20-500 Hz.

Quality of signals acquired will be seriously interfered if the high-density electromyography front-end acquisition control units are worn incorrectly: distortion of electromyograms in part of the channels will be caused if the high-density electromyography acquisition array is not tightly attached to the skin or the reference electrode is loose, which is manifested as the waveform of a signal having distortion and deformation, the amplitude of the waveform exceeding the range, and the frequency band of the signal is inconsistent with the target frequency band. By setting a quality control threshold, a damaged channel is marked to remind an operator to wear the units again, and provide a signal quality evaluation index for subsequent electromyography decomposition.

### (2) High-Density Electromyography Decoding Algorithm Module

High-density electromyography decomposition can be conducted to preprocessed electromyograms, and main components of multi-channel electromyograms can be obtained by decomposition with a FastICA method.

a. Signal Expansion

Data of a selected length is centralized and whitened, and signals are projected into a new coordinate system composed of eigenvectors by eigenvalue decomposition of a covariance matrix after the mean channel value is subtracted from each data point, so that correlation between the signals is eliminated and the signals are mutually independent.

b. Establishing Constraint Conditions to Solve a Dismixing Matrix With Maximized Non-Gaussianity Source Signals

An appropriate index is selected to measure the non-Gaussiality signals and construct an optimization problem, maximization of the non-Gaussiality is taken as a goal, and a mixing matrix is solved in constraint conditions.

c. Solving Motor Unit Action Potential by the Mixing Matrix

Mixed signals are restored to source signals by mixing matrix obtained. Further filtering and correlation inspection are conducted to the source signals restored to extract features of motor unit action potential. A clustering algorithm is introduced to combine and classify the signals obtained by separation.

Motor unit time series and firing rate information of the high-density electromyograms are output by the algorithm and can be saved to a file and exported.

1. A distributed wireless high-density surface electromyography synchronous acquisition system, comprising high-density electromyography front-end acquisition control units, a base station unit and a host computer, wherein data is transmitted between the high-density electromyography front-end acquisition control units and the base station unit through a wireless system; each high-density electromyography front-end acquisition control unit comprises a high-density electromyography acquisition array, a reference electrode and a signal transmission module; the base station unit comprises a synchronization control module, a wireless routing module and a charging and discharging module; the host computer is a client computer program, which is loaded with a signal preprocessing and display module and a high-density electromyography decoding algorithm module; and information received from the base station unit is acquired and analyzed and results are displayed by a client;
when used, the reference electrode is moistened and fixed to a skeletal muscle tendon of a human body, the high-density electromyography acquisition array is attached to a skeletal muscle belly of the human body, and the reference electrode and the high-density electromyography acquisition array are connected with the signal transmission module, respectively; after an acquisition instruction is issued by the host computer, the instruction is analyzed by the base station unit to control the high-density electromyography front-end acquisition control units to start working; skin surface electromyograms are transmitted to the signal transmission module by the high-density electromyography acquisition array, signals in the same time period are packaged by the signal transmission module and sent to the base station unit, signals transmitted by multiple high-density electromyography front-end acquisition control units are integrated by the base station unit and transmitted to the host computer through a data line in real time; and files are displayed and stored by the host computer;
wherein the signal transmission module comprises a high-density electromyography acquisition array connector, an electromyogram processing chip, a microprocessor, a six-axis acceleration sensor, a power supply control module and a battery module; the high-density electromyography acquisition array is connected by the high-density electromyography acquisition array connector to transmit original electromyograms to the signal transmission module; the electromyogram processing chip is used for amplifying and sampling, bandpass filtering and analog-to-digital conversion of the electromyograms; the microprocessor adopts a CC3235S chip integrated with a dual-band Wi-Fi low-power 5G communication chip to conduct multi-channel data integration of received high-density electromyograms and acceleration signals and real-time control of synchronization signal transmission, and finally realize lossless data processing and low-power data communication; a three-axis acceleration sensor and a three-axis gyroscope are integrated in the six-axis acceleration sensor to obtain motion data during human body electromyography data acquisition; and the power supply control module and the battery module are used for supplying power to the high-density electromyography front-end acquisition control units and protect the high-density electromyography front-end acquisition control units from electrical underload or overload, and a low voltage difference linear voltage regulator circuit is designed to reduce background noise in the electromyograms;
the skin surface electromyograms are transmitted to the signal transmission module by the high-density electromyography acquisition array and converted into digital signals with a resolution of 24 bits by the electromyogram processing chip; and the acceleration signals are transmitted to the microprocessor by the six-axis acceleration sensor, the high-density electromyograms and the acceleration signals are simultaneously received by the microprocessor, and the signals in the same time period are packaged and sent to the base station unit.

2. The distributed wireless high-density surface electromyography synchronous acquisition system according to claim 1, wherein the high-density electromyography acquisition array is composed of immersion gold contacts, a copper flat cable and a polyimide substrate, the immersion gold contacts are located on the polyimide substrate and led out by the copper flat cable; the high-density electromyography acquisition array is distributed and fixed to each skeletal muscle belly of the human body by foam double-sided adhesive tape; the maximum number of contacts in the same high-density electromyography acquisition array is 64; and the immersion gold contacts on the high-density electromyography acquisition array are arranged according to a muscle shape on the basis of ensuring a consistent spacing.

3. The distributed wireless high-density surface electromyography synchronous acquisition system according to claim 1, wherein the reference electrode is fixed to the muscle tendon of the human body and is designed into a wristband type; and one reference electrode is shared by the multiple high-density electromyography front-end acquisition control units.

4. The distributed wireless high-density surface electromyography synchronous acquisition system according to claim 1, wherein a timing pulse is sent out by the synchronization control module in the form of broadcast to inform all high-density electromyography front-end acquisition control units to conduct synchronous calibration; the synchronization control module is provided with a synchronous input port and a synchronous output port, wherein the synchronous input port is based on an signal input of an external device to start the wireless high-density surface electromyography synchronous acquisition system and complete synchronous acquisition with the external device; and when an acquisition start command is issued by the host computer, a pulse will be synchronously output by the synchronization control module to start the external device through the synchronous output port.

5. The distributed wireless high-density surface electromyography synchronous acquisition system according to claim 1, wherein the wireless routing module is used for receiving wireless signals transmitted by the high-density electromyography front-end acquisition control units and sending wireless control signals to the high-density electromyography front-end acquisition control units; and the electromyograms are transmitted to the host computer after being obtained by the wireless routing module through a wired network.

6. The distributed wireless high-density surface electromyography synchronous acquisition system according to claim 1, wherein the charging and discharging module comprises a power supply control module and a lithium ion battery, and is mainly used for charging the high-density electromyography front-end acquisition control units; and the power supply control module mainly plays the roles of reducing voltage and stabilizing current, and the lithium ion battery is used for storing electric energy to provide energy guarantee for the system when an external power supply is not available.

7. The distributed wireless high-density surface electromyography synchronous acquisition system according to claim 1, wherein the host computer is a computer program which integrates the functions of storage, calculation and display, and comprises the signal preprocessing and display module and the high-density electromyography decoding algorithm module; preprocessing of spectrum analysis and display, high and low pass filtering processing, motion artifact elimination, bad track detection and damaged data elimination of high-density data are realized by the signal preprocessing and display module, and the data is transmitted to the high-density electromyography decoding algorithm module; and preprocessed high-density electromyograms are decomposed by the high-density electromyography decoding algorithm module based on a Blind Source Separation (BSS) algorithm, and activation and discharge time series innervating motor neurons in corresponding motions are extracted.

8. A distributed wireless high-density surface electromyography synchronous acquisition device, comprising
a high-density electromyography front-end acquisition control unit which comprises a high-density electromyography acquisition array (0101), a signal transmission module (0102) and a reference electrode (0103); and the high-density electromyography acquisition array (0101) is connected with the signal transmission module (0102) through a high-density electromyography acquisition array connector (0102-1), and medical foam double-sided adhesive tape (0101-1) matched with the high-density electromyography acquisition array is arranged at the bottom of the high-density electromyography acquisition array (0101);
the signal transmission module (0102) comprises the high-density electromyography acquisition array connector (0102-1), an acquisition control unit integrated circuit board (0102-2), a charging contact (0102-3), a lithium ion rechargeable battery (0102-4), a reference electrode interface (0102-5) and a signal indicator light (0102-6); and a circuit part of the acquisition control unit integrated circuit board (0102-2) comprises an electromyogram processing chip, a six-axis acceleration sensor, a microprocessor and a power supply management module, which are designed by copper paving of a four-layer board;
a base station unit (0200) which comprises a storage box (0201), a display screen (0202), a charging case signal light (0203), a charging case (0204), a synchronous input interface (0205), a synchronous output interface (0206), a host computer communication network interface (0207), a base station unit charging interface (0208), a base station unit switch (0209), a display screen switch (0210) and base station unit power indicator lights (0211); the storage box (0201) is used for accommodating a data line and the high-density electromyography acquisition array; the display screen (0202) is used for displaying operating status information, power consumption and signal connection strength of each signal transmission module, as well as remaining power of the base station unit; the charging case signal light (0203) is used for displaying the power condition of the signal transmission module; the charging case (0204) is used for charging and accommodating the signal transmission module, and the signal transmission module can be fixed by a magnetic design in the case to ensure smooth charging of the module; the synchronous input interface (0205) is connected with an external device through a BNC q9 connector to cooperate with a peripheral device to conduct synchronous acquisition; the synchronous output interface (0206) is connected with the peripheral device to control the peripheral device to conduct synchronous acquisition; the host computer communication network interface (0207) is used for transmitting data acquired to the host computer and receiving control instructions from the host computer, which adopts a Modbus communication protocol in RS485 form and works in a full-duplex mode; the base station unit charging interface (0208) can be connected to an AC power supply to charge the base station unit; the base station unit switch (0209) is used for controlling the start and stop of the base station unit; the display screen switch (0210) is used for independently controlling the on-off of the display screen; and the base station unit power indicator lights (0211) are used for displaying the remaining power of the base station unit in real time, when four LED lights are on, it indicates that the remaining power of the base station unit is 100%, and when one LED light is off, it means that the power is reduced by 25%;
the host computer is loaded with a data preprocessing and decoding algorithm.
